(19) United States
(12) Patent Application Publication
(10) Pub. No.: US 2015/0057306 A1
(43) Pub. Date: Feb. 26, 2015

(54) COMPOSITIONS COMPRISING SCOPOLAMINE AND KETAMINE IN THE TREATMENT OF DEPRESSION

(71) Applicant: THE GENERAL HOSPITAL CORPORATION, Boston, MA (US)

(72) Inventors: Maurizio Fava, Newton, MA (US); Tracey Petryshen, Arlington, MA (US)

(21) Appl. No.: 14/388,410

(22) PCT Filed: Mar. 29, 2013
(86) PCT No.: PCT/US2013/034524

§ 371 Date: Sep. 26, 2014

Related U.S. Application Data
(60) Provisional application No. 61/618,212, filed on Mar. 30, 2012.

(51) Int. Cl. A61K 31/46; A61K 31/135

(52) U.S. Cl. CPC A61K 31/46; A61K 31/135
USPC 514/291

(57) ABSTRACT

Described are compositions and methods for administering scopolamine and ketamine in the treatment of depression (e.g., Major Depressive Disorder and Treatment-Resistant Depression).

### CROSS-REFERENCE TO RELATED APPLICATIONS

This application claims benefit under 35 U.S.C. §119(e) of U.S. Provisional Application No. 61/618,212 filed on Mar. 30, 2012, the contents of which are incorporated herein by reference in its entirety.

### FIELD OF THE INVENTION

The field of the invention relates to the treatment of depression using scopolamine and ketamine.

### BACKGROUND

Current medications for major depressive disorder (MDD) typically require weeks of treatment before clinical improvement is observed, increasing the risk of negative consequences such as suicide. Recent clinical studies have demonstrated that ketamine, an NMDA receptor antagonist, and scopolamine, a muscarinic cholinergic receptor antagonist, produce rapid antidepressant responses within hours or days of administration in depressed patients diagnosed with MDD or bipolar disorder. In addition, both drugs appear to be effective in depressed patients who are resistant to treatment (Machado-Vieira et al., 2009) (Mathew et al., 2012) (Furey and Drevets, 2006) (Drevets and Furey, 2010).

Ketamine's antidepressant action is observed after a single intravenous infusion at a dose (0.5 mg/kg) below those that induce psychotomimetic or anesthetic effects (Berman et al., 2000; Diazgranados et al., 2010; Zarate et al., 2012; Zarate et al., 2006). Significant improvement in depression ratings is typically found within 1-2 hours of ketamine administration and persists for approximately 1 week (Zarate et al., 2006). Elevated cortical excitability following ketamine infusion, as indicated by increased stimulus-evoked response in the somatosensory cortex, has been observed in responders but not non-responders, suggesting that cortical response may be a marker of ketamine's rapid antidepressant action (Cornwell et al., 2012).

Scopolamine at a low dose (0.004 mg/kg infusion) is reported to produce clinical improvement in depressed patients when assessed 3 days after treatment. However, as patients report improvement after approximately 24 hours (Furey and Drevets, 2006), clinical assessments at earlier time points after treatment may reveal more rapid effects. A recent report of a correlation between treatment response and occipital cortex BOLD response during an emotion working memory task at baseline and after scopolamine suggests that, like ketamine, brain response may be a marker of scopolamine's antidepressant effects (Furey et al., 2013).

Both scopolamine and ketamine have significant effects in rodent behavioral assays that have predictive validity for antidepressant efficacy. A single administration of scopolamine (0.1 or 0.2 mg/kg i.p.) was shown to produce reliable antidepressant effects in mice, as indicated by decreased immobility times in the tail suspension test (P<0.01 or P<0.001) and forced swim test (both P<0.001), compared to a vehicle-treated control group. These effects were not the result of non-specific motor activation, as locomotor activity was unchanged in the open field, and cognitive performance remained intact, as assessed in the passive avoidance test (Ji and Zhang, 2011). Another study reported that scopolamine (0.5-1.0 mg/kg i.p.) potentiated the antidepressant effects of desipramine (20 or 30 mg/kg i.p.) and nomifensine (2.5 or 5 mg/kg i.p.) in the forced swim test in rats (Mancinelli et al., 1988). One of the first rodent studies of antidepressant effects of ketamine (Yilmaz et al., 2002) investigated an anesthetic dose (160 mg/kg i.p.) in the forced swim test in rats. When tested 3, 7, or 10 days after ketamine treatment, immobility time was significantly decreased compared to vehicle-treated controls (i.e., an antidepressant effect). However, others have reported no effect of an anesthetic dose (80 mg/kg i.p.) immediately after treatment in rats (Li et al., 2010). An investigation of sub-anesthetic doses reported that ketamine at 10 or 15 mg/kg i.p., but not 5 mg/kg i.p., reduced forced swim test immobility time compared to a vehicle control group in rats (Garcia et al., 2008). The antidepressant effect correlated with increased levels of BDNF in the hippocampus, which clinical antidepressants are known to elevate (Chen, Dowlatshahi, et al. Biol Psych2001), suggesting a possible shared neurotrophic mechanism between ketamine and antidepressant medications.

Rodent studies of the mechanism of ketamine's antidepressant action point to rapid activation of mammalian target of rapamycin (mTOR) signaling and increased local protein synthesis in synapses, resulting in modulated synaptic function (Duman et al., 2012). Antidepressant doses of ketamine (5 and 10 mg/kg i.p.) were found to increase phosphorylation and activate several molecules in the mTOR pathway in synaptoneurosomes from rat prefrontal cortex within one hour of treatment (Li et al., 2010). These changes were followed by increased levels of pre- and postsynaptic proteins two hours after treatment, and concomitant increases in mature dendritic spines of medial PFC pyramidal neurons and serotonin-mediated cortical neurotransmission observed at 24 hours. These findings are supported in mouse models of depression, in which ketamine (3 mg/kg i.p.) exhibited an antidepressant effect in the FST within 30 minutes (Autry et al., 2011). The rapid antidepressant effect occurs alongside inhibition of eukaryotic elongation factor 2 (eEF2) kinase and dephosphorylation of eEF2, which is thought to increase translation of postsynaptic proteins including BDNF and thereby alter synaptic function (Monteggia et al., 2012). The antidepressant effect of ketamine has also been shown to require inhibition of glycogen synthase kinase 3 (GSK3) activity through increased inhibitory phosphorylation (Beurel et al., 2011), possibly through increased activity of Akt which regulates GSK3 (Duman et al., 2012).

Although the mechanism of antidepressant action of scopolamine has been less studied, scopolamine has also been reported to rapidly activate mTOR signaling (Li et al., 2011) and therefore may modulate synaptic function similar to ketamine (Drevets et al., 2012). In addition, activation of muscarinic receptors has been shown to regulate expression of NMDA receptors, and scopolamine has been reported to decrease expression of both NMDA receptor 1A and 2A in rats (Liu et al., 2004).

SUMMARY

Provided herein are compositions and methods for administering scopolamine or an active metabolites/enantiomers/isomers thereof/derivative/analogue, and ketamine or an active metabolites/enantiomers/isomers/derivative/analogue thereof in the treatment of depression with benefits including, but not limited to, significant treatment outcomes, lower dosing of each compound and improved tolerability among patients with major depressive disorder and other mood disorders.

In one aspect, provided herein is a method of treating or reducing depression in a human subject, the method comprising administering to the human subject an effective amount of ketamine or an analogue, enantiomer, isomer, or derivative thereof (e.g., about 300 mcg per kilogram of body weight per day or less) in combination with an effective amount of scopolamine or an analogue, enantiomer, isomer, or derivative thereof (e.g., about 3 mcg per kilogram of body weight per day or less), thereby treating or reducing depression in the human subject.

In another aspect, provided herein is a method of treating or reducing treatment-resistant depression in a human subject the method comprising administering to the human subject an effective amount of ketamine in combination with scopolamine, thereby treating or reducing treatment-resistant depression in the human subject. In one embodiment, the effective amount is about 300 mcg per kilogram of body weight per day or less of ketamine and about 3 mcg per kilogram of body weight per day or less of scopolamine. In another embodiment, the effective amount is 250 mcg per kilogram of body weight per day of ketamine and 2 mcg per kilogram of body weight per day of scopolamine

In another embodiment, depression comprises a disorder selected from the group consisting of major depressive disorder, mood disorder, anxiety disorder, panic disorder, post-traumatic stress disorder, dysthymic disorder, obsessive-compulsive disorder, and seasonal affective disorder.

In another embodiment, ketamine and scopolamine are administered sequentially.

In another embodiment, ketamine is administered before or after scopolamine or before and after scopolamine.

In another embodiment, scopolamine is administered before or after ketamine or before and after ketamine.

In another embodiment, ketamine and scopolamine are administered at the same time to the human subject.

In another embodiment, a composition comprising ketamine and scopolamine is administered to the human subject.

In another embodiment, 250 mcg per kilogram of body weight per day of ketamine or less and 2 mcg per kilogram of body weight per day of scopolamine or less are administered to the human subject.

In another embodiment, ketamine and/or scopolamine are injected into the blood stream, a body cavity or a subcutaneous tissue of the human subject.

Another aspect provided herein relates to a pharmaceutical composition comprising a therapeutically effective amount of ketamine, a therapeutically effective amount of scopolamine, and optionally a pharmaceutically acceptable carrier.

Also provided herein are uses of a pharmaceutical composition(s) comprising a therapeutically effective amount of ketamine, a therapeutically effective amount of scopolamine, and optionally a pharmaceutically acceptable carrier in the treatment of depression or a depressive disorder.

Other features and advantages of the methods and compositions described herein will be apparent from the detailed description, and from the claims.

### BRIEF DESCRIPTION OF THE DRAWINGS

FIG. 1 is a bar graph showing that ketamine (3mg/kg i.p.) administered to mice 30 minutes prior to testing did not significantly reduce immobility time in the FST compared to saline vehicle (p>0.05). N=6 mice/group.

FIG. 2 is a bar graph depicting scopolamine dose response in the FST in mice. Scopolamine (0.1 mg/kg i.p.) did not significantly reduce immobility time versus vehicle (p>0.05). Higher scopolamine doses (0.5 mg/kg and 1.0 mg/kg i.p.) substantially reduced immobility time (p<0.05), however this was likely due to non-specific motor activation.N=6 mice/group.

FIG. 3 is a bar graph indicating that doses of ketamine (3 mg/kg i.p.) and scopolamine (0.1 mg/kg i.p.) significantly reduce FST immobility time in mice when administered together, compared to vehicle treatment (p=0.016). Data represent two independent experiments. N=10-11 mice/group.

### DETAILED DESCRIPTION

The methods and compositions provided herein are based, in part, on the discovery that ketamine and scopolamine can be used in combination for the treatment of depression or depressive disorders. Combination therapy using ketamine and scopolamine can reduce symptoms of depression very quickly and can be used for acute therapy, for example, before clinical effects are observed with most anti-depressants, such as selective serotonin reuptake inhibitors (SSRIs). Thus, described herein are compositions and pharmaceutical formulations that comprise ketamine and/or scopolamine, and methods for using such compositions for the treatment of depression.

### Definitions

Unless otherwise defined, all technical and scientific terms used herein have the same meaning as commonly understood by one of ordinary skill in the art to which this invention belongs. In case of conflict, the present application, including definitions will control.

By “Depression” is meant a clinical entity that includes a predominantly sad or depressed mood and is accompanied by psychological and physical symptoms, often presenting as a major depressive disorder. Exemplary depressive disorders include, but are not limited to, major depressive disorder, mood disorder, anxiety disorder, panic disorder, post-traumatic stress disorder, dysthymic disorder, obsessive-compulsive disorder, and seasonal affective disorder, each of which are contemplated to be treated using the methods and compositions described herein.

By “Major Depressive Disorder” or “MDD” is meant a medical condition, lasting weeks to months to years, that includes abnormalities of affect and mood, neurovegetative functions (such as appetite and sleep disturbances), and cognition (such as inappropriate guilt and feelings of worthlessness).

By “Treatment-Resistant Depression” or “TRD” is meant any form of depression or MDD that has not responded to at least one adequate trial with antidepressant therapy.

By “an effective amount” is meant the amount of the required agents or composition comprising the agents to reduce at least one symptom of depression relative to an untreated patient. The effective amount of composition(s) for therapeutic treatment of a disease varies depending upon the manner of administration, the age, body weight, and general health of the subject. Ultimately, the attending physician will decide the appropriate amount and dosage regimen.

The term “reduced” or “reduce” or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90%, at least 95%, at least 99% or up to and including a 100% decrease (i.e. substantially absent or below levels of detection), or any decrease between 10-100% as compared to a reference level, as that term is defined herein. As used herein, the term “standard” or “reference” can simply be a reference that defines a baseline for comparison, such as a healthy individual(s) not suffering from depression. A “subject” is a vertebrate, including any member of the class Mammalia, including humans, domestic and farm animals, and zoo, sports or pet animals, such as mouse, rabbit, pig, sheep, goat, cattle and higher primates. In one embodiment, the subject is a human.

As used herein, the terms “treat”, “treatment” or “treating” used in reference to a disease or disorder (e.g., depression) refers to measures that delay the onset, reverse, alleviate, ameliorate, decrease, inhibit, or slow down the progression or severity of a condition or symptom associated with a disease or disorder. In one embodiment, the term “treating” includes reducing or alleviating at least one adverse effect or symptom of a condition, disease or disorder associated with depression, such as, but not limited to abnormalities of affect and mood, neurovegetative functions (such as appetite and sleep disturbances), and cognition (such as inappropriate guilt and feelings of worthlessness). Treatment is generally “effective” if one or more symptoms or clinical markers are reduced as that term is defined herein. Alternatively, treatment is “effective” if the progression of a disease is reduced or halted. That is, “treatment” includes not just the improvement of symptoms or markers, but also a cessation or at least slowing of progress or worsening of symptoms that would be expected in the absence of treatment. In some embodiments of the aspects described herein, the symptoms or a measured parameter of depression are alleviated by at least 5%, at least 10%, at least 20%, at least 30%, at least 40%, at least 50%, at least 60%, at least 70%, at least 80%, or at least 90%, upon administration of agents, as compared to a control or non-treated subject. Thus, one of skill in the art realizes that a “treatment” can improve the disease condition, but need not be a complete cure for the disease.

The phrase “combination therapy” (or “co-therapy”) embraces the administration of ketamine (or an analogue, derivative, enantiomer, active metabolite, or salt thereof) and scopolamine (or an analogue, derivative, enantiomer, active metabolite, or salt thereof) as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients (such as, but not limited to, a second and different agent for treatment of low ketamine levels, and/or for treatment of prostate cancer etc.,) and non-drug therapies (such as, but not limited to, surgery or radiation treatment).

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation, for example the carrier does not decrease the impact of the agent on the treatment. In other words, a carrier is pharmaceutically inert.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

Other than in the operating examples, or where otherwise indicated, all numbers expressing quantities of ingredients or reaction conditions used herein should be understood as modified in all instances by the term “about.” The term “about” when used in connection with percentages can mean ±1%.

In this application and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 18th Edition, published by Merck Research Laboratories, 2006 (ISBN 0-911910-18-2); Robert S. Porter et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006. Definitions of common terms in molecular biology are found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987); Current Protocols in Molecular Biology (CPMB) (Fred M. Ausubel, et al. ed., John Wiley and Sons, Inc.), Current Protocols in Protein Science (CPPS) (John E. Coligan, et. al., ed., John Wiley and Sons, Inc.) and Current Protocols in Immunology (CPI) (John E. Coligan, et. al., ed. John Wiley and Sons, Inc.), which are all incorporated by reference herein in their entireties.

### Depression and Related Disorders

Depression can be characterized by sadness, loss of interest in activities, and decreased energy. Other symptoms include loss of confidence and self- esteem, inappropriate guilt, thoughts of death and suicide, diminished concentration, and disturbance of sleep and appetite. A variety of somatic symptoms can also be present. Though depressive feelings are common, especially after experiencing setbacks in life, depressive disorder is diagnosed only when the symptoms reach a threshold and last at least two weeks. Depression can vary in severity from mild to very severe, and includes unipolar and bipolar depression, as well as seasonal affective disorder (SAD). Depression is typically also characterized into eight basic dimensions i.e., Pessimism, Weak Concentration, Sleep Problems, Anhedonia, Fatigue, Loneliness, Low Self- esteem, and Somatic Complaints to define the profile of children's and adolescents' depression. Depression can occur as an idiopathic disease (with no somatic disease associated with it), or it can be a psychiatric symptom of a somatic disorder, especially a number of neurodegenerative disorders.

Scales known in the art to be administered in assessing levels of depression include:

(1) Hamilton Depression Rating Scale 28-Item: primary outcome measure (Hamilton M. J., Neurol Neurosurg Psychiatry 1960. 23:56-62; Hamilton M., Br J Social Clin Psychology 1967. 6:278-296).

(2) Columbia-Suicide Severity Rating Scale (Posner K. et al., Am J Psychiatry. 2011 Dec; 168(12):1266-77).

(3) Clinical Global Improvement Scale—Severity and Improvement (Guy W. Clinical Global Impression (CGI) ECDEU Assessment manual for Psychopharmacology. Rockville, Md.: U.S. Dept Health Education and Welfare 1976).

(4) Quick Inventory of Depressive Symptoms, Self-Report version (Trivedi M. H. et al., Psychol Med. 2004 Jan; 34(1):73-82).

(5) Concise Health Risk Tracking (Trivedi M. H. et al., J Clin Psychiatry. 2011 Jun; 72(6):757-64).

(6) MGH Cognitive and Physical Functioning Questionnaire (Fava M. et al., Psychother Psychosom. 2009; 78(2): 91-7).

(7) Quality of Life Enjoyment and Satisfaction Questionnaire (Endicott J. et al., Psychopharmacol Bull. 1993; 29(2): 321-6).

(8) Brief Psychiatric Rating Scale (Andersen J. et al. Psychopathology. 1989; 22(2-3): 168-76; Hafkenscheid A., Acta Psychiatr Scand. 1991 Sep; 84(3): 294-300).

(9) Only at baseline and end of Phase 3 - Neurocognitive Test Battery immediate and delayed verbal recall, speed of comprehension, digit span forward and backward, N-back test, and trail-making task (Trandafir A. et al., Schizophr Res. 2006 Jan 31; 81(2-3): 217-26).

### Compositions and Methods of Treatment

Provided herein are compositions (e.g., pharmaceutical compositions) and formulations comprising scopolamine (or active metabolites/enantiomers/isomers/derivatives/analogues thereof) and/or ketamine (or its active metabolites/enantiomers/isomers/derivatives/analogues thereof) for the treatment of depression. While the terms “ketamine” and “scopolamine” are used throughout the specification, it is contemplated herein that such terms include an active metabolite, enantiomer, isomer, derivative, analogue or salt thereof of ketamine and/or an active metabolite, enantiomer, isomer, derivative, analogue or salt thereof of scopolamine.

In alternative embodiments, the compositions described herein are formulated with a pharmaceutically acceptable carrier. In alternative embodiments, the pharmaceutical compositions and formulations of the invention can be administered parenterally, topically, orally or by local administration, such as by aerosol, intranasally, or transdermally. The pharmaceutical compositions can be formulated in any way and can be administered in a variety of unit dosage forms depending upon the condition or disease (e.g., depression) and the degree of illness, the general medical condition of each patient, the resulting preferred method of administration and the like. Details on techniques for formulation and administration of pharmaceuticals are well described in the scientific and patent literature, see, e.g., the latest edition of Remington's Pharmaceutical Sciences, Maack Publishing Co, Easton Pa. (“Remington's”).

Compositions as described herein can be administered alone or as a component of a pharmaceutical formulation. The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Compositions as described herein include those suitable for intradermal, inhalation, oral/nasal, topical, parenteral, rectal, and/or intravaginal administration. The formulations can be presented in unit dosage form and can be prepared by any methods well known in the art of pharmacy. The amount of active ingredient (e.g., scopolamine or its active metabolites/enantiomers/isomers and/or ketamine or its active metabolites/enantiomers/isomers) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g., an antidepressant effect.

Pharmaceutical formulations of the compositions described herein can be prepared according to any method known to the art for the manufacture of pharmaceuticals. Such drugs can contain sweetening agents, flavoring agents, coloring agents and preserving agents. A formulation can be admixed with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., scopolamine and/or ketamine) in an admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In one embodiment, oil-based pharmaceuticals are used for administration. Oil-based suspensions can be formulated by suspending an active agent (e.g., scopolamine and/or ketamine) in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

Pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate and/or an ethoxylated sorbitan trioleate.

The pharmaceutical formulations described herein can also be administered by intranasal, intraocular and intravaginal routes including suppositories, insufflation, powders and aerosol formulations (for examples of steroid inhalants, see e.g., Rohatagi (1995) J. Clin. Pharmacol. 35:1187-1193; Tjwa (1995) Ann. Allergy Asthma Immunol 75:107-111). Suppository formulations can be prepared by mixing the drug with a suitable non-irritating excipient which is solid at ordinary temperatures but liquid at body temperatures and will therefore melt in the body to release the drug. Such materials can be cocoa butter and polyethylene glycols.

The pharmaceutical compounds comprising scopolamine and/or ketamine can be delivered transdermally, by a topical route, formulated as applicator sticks, solutions, suspensions, emulsions, gels, creams, ointments, pastes, jellies, paints, powders, and aerosols.

The pharmaceutical formulations described herein can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

Pharmaceutical formulations can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose, any bland fixed oil can be employed including synthetic mono- or diglycerides. In addition, fatty acids such as oleic acid can likewise be used in the preparation of injectables. These solutions are sterile and generally free of undesirable matter. These formulations can be sterilized by conventional, well known sterilization techniques. The formulations can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and are selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

The pharmaceutical compounds and formulations described herein can be lyophilized. Thus, provided herein are stable, lyophilized formulations comprising a composition as described herein, which can be made by lyophilizing a solution comprising a pharmaceutical of the invention and a bulking agent, e g., mannitol, trehalose, raffinose, and sucrose or mixtures thereof.

The compositions and formulations comprising ketamine and/or scopolamine can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587.

The amount of pharmaceutical composition adequate to conduct the methods described herein is an effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on for example: the dosage and frequency as required and tolerated by the patient, the degree and amount of antidepressant efficacy generated after each administration, and the like. The formulations should provide a sufficient quantity of active agent (scopolamine and/or ketamine) to effectively treat, prevent or ameliorate at least one symptom of depression.

In alternative embodiments, pharmaceutical formulations for intravenous administration are in a daily amount of between about 300 mcg or less, including 275, 250, 200, 175, 150, 125, 100, 75, 50 or 25 mcg per kilogram of body weight per day for ketamine and about 3 mcg or less, including 2.75, 2.5, 2.0, 1.75, 1.5, 1.25, 1.0, 0.75, 0.50, 0.25 or 0.20 mcg per kilogram of body weight per day for scopolamine.

In some embodiments, the dose of ketamine is at least 25 mcg per kilogram, at least 30 mcg per kilogram, at least 40 mcg per kilogram, at least 50 mcg per kilogram, at least 60 mcg per kilogram, at least 70 mcg per kilogram, at least 75 mcg per kilogram, at least 80 mcg per kilogram, at least 90 mcg per kilogram, at least 100 mcg per kilogram, at least 125 mcg per kilogram, at least 150 mcg per kilogram, at least 175 mcg per kilogram, at least 200 mcg per kilogram, at least 225 mcg per kilogram, at least 250 mcg per kilogram, at least 275 mcg per kilogram, or at least 300 mcg per kilogram or more.

In other embodiments, the dose of scopolamine is at least 0.20 mcg per kilogram, at least 0.25 mcg per kilogram, at least 0.5 mcg per kilogram, at least 0.75 mcg per kilogram, at least 1.0 mcg per kilogram, at least 1.25 mcg per kilogram, at least 2.0 mcg per kilogram, at least 2.5 mcg per kilogram, at least 2.75 mcg per kilogram, at least 3.0 mcg per kilogram or more.

In some embodiments, the daily range of ketamine administered is from 25-50mcg per kilogram, from 25-75 mcg per kilogram, from 25-100 mcg per kilogram, from 25-125 mcg per kilogram, from 25-150 mcg per kilogram, from 25-175 mcg per kilogram, from 25-200 mcg per kilogram, from 25-250 mcg per kilogram, from 25-275 mcg per kilogram, from 25-300 mcg per kilogram, from 275-300 mcg per kilogram, from 250-300 mcg per kilogram, from 200-300 mcg per kilogram, from 175-300 mcg per kilogram, from 150-300 mcg per kilogram, from 125-300 mcg per kilogram, from 100-300 mcg per kilogram, from 75-300 mcg per kilogram, from 50-300 mcg per kilogram, from 50-75 mcg per kilogram, from 75-100 mcg per kilogram, from 100-125 mcg per kilogram, from 100-150 mcg per kilogram, from 150-175 mcg per kilogram, from 100-200 mcg per kilogram, or from 275-300 mcg per kilogram.

In some embodiments, the daily range of scopolamine administered is from 0.2-3.0 mcg per kilogram, from 0.2-2.75 mcg per kilogram, from 0.2-2.5 mcg per kilogram, from 0.2-2.0 mcg per kilogram, from 0.2-1.5 mcg per kilogram, from 0.2-1.0 mcg per kilogram, from 0.2-0.75 mcg per kilogram, from 0.2-0.5 mcg per kilogram, from 0.2-0.25 mcg per kilogram, from 2.75-3.0 mcg per kilogram, from 2.5-3.0 mcg per kilogram, from 2.0-3.0 mcg per kilogram, from 1.75-3.0 mcg per kilogram, from 1.5-3.0 mcg per kilogram, from 1.0-3.0 mcg per kilogram, from 0.75-3.0 mcg per kilogram, from 0.5-3.0 mcg per kilogram, from 0.25-3.0 mcg per kilogram, from 0.5-3.0mcg per kilogram, from 0.5-0.75 mcg per kilogram, from 0.75-1.0 mcg per kilogram, from 1.0-1.25 mcg per kilogram, from 1.0-1.5 mcg per kilogram, from 1.50-1.75 mcg per kilogram, from 1.00-2.00 mcg per kilogram, or from 2.75-3.00mcg per kilogram.

Preferably, ketamine can be administered at an initial dose of 250 mcg/kg and scopolamine can be administered at a dose of 2 mcg/kg. In specific embodiments, ketamine is administered at a dose of 250 mcg/kg or less over a 45-minute period by a controlled infusion (e.g., using a syringe with a Medafusion pump) and scopolamine is administered at a dose of 2 mcg/kg or less over 15 minutes by a controlled infusion. Subjects can receive an infusion of scopolamine and saline, ketamine and saline or both active drugs together. Higher dosages (than those mentioned above) can be used for oral administration in contrast to administration into the blood stream, into a body cavity or into a lumen of an organ. Substantially higher dosages can be used in topical or oral administration or administering by powders, spray or inhalation. Actual methods for preparing parenterally or non-parenterally administrable formulations are known or apparent to those skilled in the art.

It will also be appreciated that the compounds and pharmaceutical compositions described herein can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another antidepressant or mood stabilizing agent), or they may achieve different effects (e.g., control of any adverse effects).

The methods described herein can further comprise co-administration of the compositions comprising ketamine and/or scopolamine with other drugs or pharmaceuticals, e.g., compositions for treating depression. For example, the methods and/or compositions and formulations of the invention can be co-administered with a tricyclic antidepressant, a benzodiazepine, an atypical antipsychotics, an anticonvulsant, or a selective serotonin reuptake inhibitor, including, but not limited to, fluoxetine, citalopram, paroxetine, escitalopram, sertraline, and any combinations thereof.

Useful combination therapies will be understood and appreciated by those of skill in the art. Potential advantages of such combination therapies include the ability to use less of each of the individual active ingredients to minimize toxic side effects, synergistic improvements in efficacy, improved ease of administration or use, and/or reduced overall expense of compound preparation or formulation.

In some embodiments, a combination of ketamine, or an analogue, derivative thereof with scopolamine, or an analogue thereof, as disclosed herein can be used in combination with other agents to maximize the effect of the compositions administered in an additive or synergistic manner.

The effective amount of the compositions described herein can be administered to a selected human subject as a single daily dose, or alternatively, in more than one divided doses per day via any suitable administration route, e.g., oral administration.

In some embodiments, a composition as disclosed herein for use in the methods as disclosed herein comprises ketamine or an analogue or derivative or salt thereof, for use in combination with scopolamine or an analogue or enantiomer or active metabolite or derivative or salt thereof. In some embodiments, a composition comprising ketamine or an analogue enantiomer or active metabolite or derivative or salt thereof, for use in combination with scopolamine or an analogue or derivative or enantiomer or active metabolite or salt thereof comprises at least about 80%, or at least about 85%, or at least about 90%, or at least about 92%, or at least about 95%, or at least about 97%, or at least about 98%, or at least about 99%, or at least about 99.5%, or at least about 99.8% or more than 99.8% of one or more of ketamine or an analogue or derivative or enantiomer or active metabolite or salt thereof and scopolamine or an analogue or derivative or enantiomer or active metabolite or salt thereof.

As used herein, the term “composition” is intended to encompass a product comprising the specified ingredients in the specified amounts, as well as any product which results, directly or indirectly, from combinations of the specified ingredients in the specified amounts.

Administration of a composition comprising a combination of ketamine, analogue or salt thereof and scopolamine or an analogue or derivative or salt thereof as disclosed herein may be by oral, parenteral, sublingual, rectal, or enteral administration, or pulmonary absorption or topical application. Direct administration of a composition comprising a combination of ketamine, analogue or salt thereof and scopolamine or an analogue or derivative or salt thereof as disclosed herein to a subject can be by oral, parenteral, sublingual, rectal such as suppository or enteral administration, or by pulmonary absorption or topical application. Parenteral administration may be by intravenous (IV) injection, subcutaneous (s.c.) injection, intramuscular (i.m) injection, intra-arterial injection, intrathecal (i.t.) injection, intra-peritoneal (i.p) injection, or direct injection or other administration to the subject.

In addition to a composition comprising a combination of ketamine, analogue or salt thereof and scopolamine or an analogue or derivative or salt thereof as disclosed herein, such compositions can optionally contain pharmaceutically-acceptable carriers and other ingredients known to facilitate administration and/or enhance uptake (e.g., saline, dimethyl sulfoxide, lipid, polymer, affinity-based cell specific-targeting systems). In some embodiments, a composition comprising a combination of ketamine, analogue or salt thereof and scopolamine or an analogue or derivative or salt thereof as disclosed herein and/or salts thereof can be incorporated in a gel, sponge, or other permeable matrix (e.g., formed as pellets or a disk) and placed in proximity to the endothelium for sustained, local release. In some embodiments, a composition comprising a combination of ketamine, analogue or salt thereof and scopolamine or an analogue or derivative or salt thereof as disclosed herein and/or salts thereof can be administered in a single dose or in multiple doses which are administered at different times.

In some embodiments, the ketamine, or ketamine derivative or analogue or salt thereof, and scopolamine or an analogue or derivative or salt thereof as disclosed herein can be provided in a therapeutic composition so that the preferred amounts of the ketamine agent and scopolamine agent are supplied by a single dosage, for example a single capsule enabling ketamine, (or a ketamine derivative or analogue or salt thereof), and scopolamine (or an analogue or derivative or salt thereof) to be administered to a subject at about the same time.

In some embodiments of the invention, ketamine, (or a ketamine derivative, analogue or salt thereof), and scopolamine (or an analogue or derivative or salt thereof) can be administered substantially simultaneously, meaning that both agents can be provided in a single dosage, for example by mixing the agents and incorporating the mixture into a single capsule or within a short time of each other. In alternative embodiments, ketamine, (or a ketamine derivative, analogue or salt thereof), scopolamine (or an analogue or derivative or salt thereof) can be administered substantially simultaneously by administration in separate dosages within a short time period, for example within one hour or less, 45 minutes or less, 30 minutes or less, 15 minutes or less, 10 minutes or less, 5 minutes or less and all time periods in between. Alternatively, ketamine, (or a ketamine derivative, analogue or salt thereof), and scopolamine (or an analogue or derivative or salt thereof) can be administered sequentially, meaning that separate dosages, and possibly even separate dosage forms of ketamine, (or a ketamine derivative, analogue or salt thereof), and scopolamine (or an analogue or derivative or salt thereof) can be administered at separate times, for example on a staggered schedule but with equal frequency of administration of each of the ketamine, (or a ketamine derivative, analogue or salt thereof), and scopolamine (or an analogue or derivative or salt thereof). Of course, it is also possible that ketamine, (or a ketamine derivative, analogue or salt thereof), can be administered either more or less frequently than the one or more scopolamine agent. Different agents have different half-lives, thus one can stagger schedules and still maintain both agents being effective in an individual. In any case, it is preferable that, among successive time periods of a sufficient length, for example one day, the weight ratio of ketamine, (or a ketamine derivative, analogue or salt thereof), are administered to the weight ratio of the scopolamine agent administered remains constant.

In some embodiments, ketamine, (or a ketamine derivative, analogue or salt thereof), and scopolamine (or an analogue or derivative or salt thereof) can be administered sequentially, for example the two agents can be administered within about 1 hour or more of each other, as long as they are both biologically active within the same time period. For example, the time between administration of the ketamine, (or a ketamine derivative, analogue or salt thereof), and scopolamine (or an analogue or derivative or salt thereof) can vary depending on the half-life of each of the agents. For example a longer time period can occur between administration of ketamine, (or a ketamine derivative, analogue or salt thereof), and scopolamine (or an analogue or derivative or salt thereof), if both agents have long half-lives, as compared to a shorter time period between administration of each agent if both agents have short half-lives. Alternatively, the first agent administered, for example ketamine, (or a ketamine derivative, analogue or salt thereof), can be administered in a time-release capsule to release the biologically active ketamine agent at a certain period after administration, or ketamine, (or a ketamine derivative, analogue or salt thereof), can be administered as a pro-drug that takes a certain time period to be metabolized to become the biologically active compound, and where in both situations the ketamine becomes biologically active at a time period which coincides with administration and biological activity of the scopolamine (or an analogue or derivative or salt thereof).

In alternative embodiments, ketamine, (or a ketamine derivative, analogue or salt thereof), and scopolamine (or an analogue or derivative or salt thereof) can be administered sequentially, for example, one can administer a ketamine to a subject followed by at least one scopolamine agent , then a ketamine agent, and so forth, so that the subject is administered, in an alternating regimen, doses of ketamine (or a ketamine derivative, analogue or salt thereof) followed by a dose of scopolamine (or an analogue or derivative or salt thereof), or vice versa.

In alternative embodiments, a subject is administered one agent continuously and administered the other agent in repeated doses. By way of an example but not as a limitation, a subject can be continuously administered ketamine (or a ketamine derivative, analogue or salt thereof) by any suitable means such as a transdermal patch or other continuous administration method such as catheterization or by pump administration and administered scopolamine (or an analogue or derivative or salt thereof) or vice versa at regular intervals, for example but not limited to daily, twice a day, twice a week, monthly etc. by any suitable means known by persons of ordinary skill in the art and disclosed herein to keep the agents active in an individual.

In alternative embodiments, a subject is administered ketamine, (or a ketamine derivative, analogue or salt thereof), and scopolamine (or an analogue or derivative or salt thereof) by pulse chase schedules. For example, a subject is administered one agent, such as a scopolamine agent for a brief period of time (the pulse) and then a subject is administered the other agent, such as a ketamine agent for a longer period (the chase), or vice versa. In such embodiments, a subject can be administered varying amounts of each agent for each pulse-chase administration regimen. The pulse-chase regime can be switched so that the subject is administered a ketamine agent for a limited period of time, followed by administration of the scopolamine agent for a longer period of time.

In some embodiments, a subject is administered varying amounts of each agent, for example varying amounts of ketamine (or a ketamine derivative, analogue or salt thereof) and varying amounts of scopolamine (or an analogue or derivative or salt thereof).

Daily dosages can vary within wide limits and will be adjusted to the subject requirements in each particular case. In general, for administration to adults, an appropriate daily dosage has been described above, although the limits that were identified as being preferred can be exceeded if necessary. The daily dosage can be administered as a single dosage or in divided dosages. Various delivery systems include capsules, tablets, and gelatin capsules, for example.

Any suitable route and any combination of routes of administration can be employed for providing a subject with an effective dosage of a combined therapy of the present invention. For example, oral, rectal, transdermal, parenteral (subcutaneous, intramuscular, intravenous), intrathecal, and like forms of administration can be employed. Dosage forms include tablets, troches, dispersions, suspensions, solutions, capsules, patches, and the like.

The compositions as described herein are administered and dosed in accordance with good medical practice, taking into account the clinical condition of the individual subject, the site and method of administration, scheduling of administration, subject age, sex, body weight and other factors known to medical practitioners.

Pharmaceutical compositions comprising a combination of ketamine, analogue or salt thereof and scopolamine (or an analogue or derivative or salt thereof) as disclosed herein and/or salts thereof can be administered by any known route. By way of example, Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. and/or salts thereof can be administered by a mucosal, pulmonary, topical, or other localized or systemic route (e.g., enteral and parenteral).

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection, infusion and other injection or infusion techniques, without limitation. The phrases “systemic administration,” “administered systemically”, “peripheral administration” and “administered peripherally” as used herein mean the administration of the agents as disclosed herein such that it enters the animal's system and, thus, is subject to metabolism and other like processes, for example, subcutaneous administration.

Preparations of a composition comprising a combination of ketamine, analogue or salt thereof and scopolamine (or an analogue or derivative or salt thereof) are administered in effective amounts. An effective amount is that amount of a pharmaceutical preparation that alone, or together with further doses, stimulates the desired response. Typically an effective amount of such ketamine compounds and the amount of scopolamine (or an analogue or derivative or salt thereof) can be determined by an ordinary physician, or in clinical trials, establishing an effective dose for a test population versus a control population in a blind study, where the effective dose results in beneficial antidepressant effects of ketamine in combination with scopolamine (or an analogue or derivative or salt thereof).

### Oral Formulations

In some embodiments, administration of a composition comprising a combination of ketamine, analogue or salt thereof and scopolamine (or an analogue or derivative or salt thereof) as disclosed herein is in an oral formulation. Alternatively, in some embodiments, compositions comprising a combination of ketamine, analogue or salt thereof and scopolamine (or an analogue or derivative or salt thereof) can also be administered to the nasal passages as a spray. Sprays also provide immediate access to the pulmonary system and are the preferable methods for administering compositions immediately to the subject. Access to the gastrointestinal tract is gained using oral, enema, or injectable forms of administration. For example, administration of the compositions comprising a combination of ketamine, analogue or salt thereof and scopolamine (or an analogue or derivative or salt thereof) as disclosed herein and/or salts thereof to a subject is preferably oral. As a result, the subject can undergo administration of a composition comprising a combination of ketamine, analogue or salt thereof and scopolamine (or an analogue or derivative or salt thereof) at home.

As indicated above, orally active compositions comprising a combination of ketamine, analogue or salt thereof and scopolamine (or an analogue or derivative or salt thereof) as disclosed herein are preferred for at least a portion of the cycle of therapy, as oral administration is usually the safest, most convenient, and economical mode of drug delivery. Consequently, compositions as disclosed herein comprising a combination of ketamine, analogue or salt thereof and scopolamine (or an analogue or derivative or salt thereof) as disclosed herein thereof can be modified to increase their oral bioavailability by reducing or eliminating their polarity. This can often be accomplished by formulating a composition with a complimentary reagent that neutralizes its polarity, or by modifying the compound with a neutralizing chemical group. Oral bioavailability can be challenging because drugs are exposed to the extremes of gastric pH and gastric enzymes. Accordingly, challenges associated with oral bioavailability can be overcome by modifying the molecular structure to be able to withstand very low pH conditions and resist the enzymes of the gastric mucosa such as by neutralizing an ionic group, by covalently bonding an ionic interaction, or by stabilizing or removing a disulfide bond or other relatively labile bond.

In some embodiments, an oral formulation of a composition comprising a combination of ketamine, analogue or salt thereof and scopolamine (or an analogue or derivative or salt thereof) as disclosed herein can comprise trappsol and/or captisol for stability, or alternatively cyclodextrin. In some embodiments, an oral formulation a composition comprising a combination of ketamine, analogue or salt thereof and scopolamine (or an analogue or derivative or salt thereof) as disclosed herein comprises a preservative, for example, methylparaben, which can be used for example at a concentration of about 0.25% for the syrup formulation in the pH range of 6-7. Applicants recommend a preservative challenge test to be conducted at a later stage and a variety of different timepoints to determine the optimal concentration of methylparaben based on the results of the preservative challenge test.

In some embodiments, an oral formulation of a composition comprising a combination of ketamine, analogue or salt thereof and scopolamine (or an analogue or derivative or salt thereof) as disclosed herein thereof can also comprise a sweetener, for example, it can be formulated as a syrup using any sweetener commonly known to one of ordinary skill in the art, and in different combinations and percentage of the formulation. Exemplary sweeteners include, but are not limited to, Sucrose syrup, High Fructose Corn syrup, Sodium saccharin, Aspartame, Acesulfame and Sucralose. In some embodiments, an oral formulation of a combination of ketamine, analogue or salt thereof and scopolamine (or an analogue or derivative or salt thereof) as disclosed herein comprises at least one sweetener(s) or a combination of any sweeteners and a stabilizer, e.g., but not limited to Trappsol.

In some embodiments, an oral formulation of a composition comprising a combination of ketamine, analogue or salt thereof and scopolamine (or an analogue or derivative or salt thereof) as disclosed herein comprises at least one, or any combination of High Fructose Corn syrup, Sodium saccharin, Aspartame, Acesulfame or Sucralose.

In some embodiments, an oral formulation of a composition comprising a combination of ketamine, analogue or salt thereof and scopolamine (or an analogue or derivative or salt thereof) as disclosed herein can also comprise a flavor, for example, any flavor known to persons of ordinary skill in the art, for example, but without limitation, Cherry, Grape, Lemon, Pineapple, Orange, Menthol, Chocolate, Mint, Chocolate mint

### Enteric Coated Formulation

In some embodiments, a composition comprising a combination of ketamine, analogue or salt thereof and scopolamine (or an analogue or derivative or salt thereof) as disclosed herein can be formulated as tablets, for oral and/or enteral administration in accordance with conventional procedures employing solid carriers well-known in the art. Capsules employed for oral formulations to be used with the methods as described herein can be made from any pharmaceutically acceptable material, such as gelatin or cellulose derivatives. Sustained release oral delivery systems and/or enteric coatings for orally administered dosage forms are also contemplated, such as those described in U.S. Pat. No. 4,704,295, “Enteric Film-Coating Compositions,” issued Nov. 3, 1987; U.S. Pat. No. 4,556,552, “Enteric Film—Coating Compositions,” issued Dec. 3, 1985; U.S. Pat. No. 4,309,404, “Sustained Release Pharmaceutical Compositions,” issued Jan. 5, 1982; and U.S. Pat. No. 4,309,406, “Sustained Release Pharmaceutical Compositions,” issued Jan. 5, 1982, which are all incorporated herein in their entirety by reference.

Accordingly in some embodiments, oral formulations of a composition comprising a combination of ketamine, analogue or salt thereof and scopolamine (or an analogue or derivative or salt thereof) as disclosed herein can be in the form of a tablet formulation, for example, a tablet an enteric polymer casing. An example of such a preparation can be found in WO2005/021002, which is incorporated herein in its entirety by reference. The active material in the core can be present in a micronized or solubilized form. In addition to active materials the core can contain additives conventional to the art of compressed tablets. Appropriate additives in such a tablet can comprise diluents such as anhydrous lactose, lactose monohydrate, calcium carbonate, magnesium carbonate, dicalcium phosphate or mixtures thereof; binders such as microcrystalline cellulose, hydroxypropylmethylcellulose, hydroxypropyl-cellulose, polyvinylpyrrolidone, pre-gelatinised starch or gum acacia or mixtures thereof; disintegrants such as microcrystalline cellulose (fulfilling both binder and disintegrant functions) cross-linked polyvinylpyrrolidone, sodium starch glycollate, croscarmellose sodium or mixtures thereof; lubricants, such as magnesium stearate or stearic acid, glidants or flow aids, such as colloidal silica, talc or starch, and stabilizers such as desiccating amorphous silica, coloring agents, flavors etc. In some embodiments, a tablet comprises lactose as diluent. When a binder is present, it is preferably hydroxypropylmethyl cellulose. In some embodiments, a tablet comprises magnesium stearate as lubricant. In some embodiments, a tablet comprises croscarmellose sodium as disintegrant, or can comprise a microcrystalline cellulose.

Examples of solid carriers include starch, sugar, bentonite, silica, and other commonly used carriers. Further non-limiting examples of carriers and diluents that can be used in the formulations as described herein include saline, syrup, dextrose, and water.

In some embodiments, a diluent can be present in a range of 10-80% by weight of the core. The lubricant can be present in a range of 0.25-2% by weight of the core. The disintegrant can be present in a range of 1-10% by weight of the core. Microcrystalline cellulose, if present, can be present in a range of 10-80% by weight of the core.

In some embodiments, the active ingredient, e.g., a combination of ketamine, analogue or salt thereof and/or scopolamine (or an analogue or derivative or salt thereof) comprises between 10 and 50% of the weight of the core, more preferably between 15 and 35% of the weight of the core (calculated as free base equivalent). The core can contain any therapeutically suitable dosage level of the active ingredient e.g., a combination of ketamine, analogue or salt thereof and/or scopolamine (or an analogue or derivative or salt thereof), but preferably contains up to 150 mg as free base of the active ingredient. In some embodiments, the core contains 20, 30, 40, 50, 60, 80 or 100 mg as free base of the active ingredient. The active ingredient e.g., a combination of ketamine, analogue or salt thereof and/or scopolamine (or an analogue or derivative or salt thereof) can be present as the free base, or as any pharmaceutically acceptable salt. If the active ingredient e.g., a combination of ketamine, analogue or salt thereof and an scopolamine (or an analogue or derivative or salt thereof) present as a salt, the weight is adjusted such that the tablet contains the desired amount of active ingredient, calculated as free base of the salt.

In some embodiments, the core can be made from a compacted mixture of its components. The components can be directly compressed, or can be granulated before compression. Such granules can be formed by a conventional granulating process as known in the art. In an alternative embodiment, the granules can be individually coated with an enteric casing, and then enclosed in a standard capsule casing.

In some embodiments, the core can be surrounded by a casing that comprises an enteric polymer. Examples of enteric polymers are cellulose acetate phthalate, cellulose acetate succinate, methylcellulose phthalate, ethylhydroxycellulose phthalate, polyvinylacetate pthalate, polyvinylbutyrate acetate, vinyl acetate-maleic anhydride copolymer, styrene-maleic mono-ester copolymer, methyl acrylate-methacrylic acid copolymer or methacrylate-methacrylic acid-octyl acrylate copolymer. These can be used either alone or in combination, or together with other polymers than those mentioned above. The casing can also include insoluble substances which are neither decomposed nor solubilized in living bodies, such as alkyl cellulose derivatives such as ethyl cellulose, crosslinked polymers such as styrene-divinylbenzene copolymer, polysaccharides having hydroxyl groups such as dextran, cellulose derivatives which are treated with bifunctional crosslinking agents such as epichlorohydrin, dichlorohydrin or 1, 2-, 3, 4-diepoxybutane. The casing can also include starch and/or dextrin.

In some embodiments, enteric coating materials are the commercially available EUDRAGIT® enteric polymers such as EUDRAGIT® L, EUDRAGIT® S and EUDRAGIT® NE, used alone or with a plasticiser. Such coatings are normally applied using a liquid medium, and the nature of the plasticiser depends upon whether the medium is aqueous or non-aqueous. Plasticisers for use with aqueous medium include propylene glycol, triethyl citrate, acetyl triethyl citrate or CITROFLEX® or CITROFLEX® A2. Non-aqueous plasticisers include these, and also diethyl and dibutyl phthalate and dibutyl sebacate. A preferred plasticiser is Triethyl citrate. The quantity of plasticiser included will be apparent to those skilled in the art.

In some embodiments, a casing can also include an anti-tack agent such as talc, silica or glyceryl monostearate. In some embodiments, an anti-tack agent is glyceryl monostearate. Typically, the casing can include around 5-25 wt % Plasticiser and up to around 50 wt % of anti-tack agent, preferably 1-10 wt % of anti-tack agent.

If desired, a surfactant can be included to aid with forming an aqueous suspension of the polymer. Many examples of possible surfactants are known to the person skilled in the art. Preferred examples of surfactants are polysorbate 80, polysorbate 20, or sodium lauryl sulphate. If present, a surfactant can form 0.1-10% of the casing, preferably 0.2-5% and particularly preferably 0.5-2%

In one embodiment, there is a seal coat included between the core and the enteric coating. A seal coat is a coating material that can be used to protect the enteric casing from possible chemical attack by any alkaline ingredients in the core. The seal coat can also provide a smoother surface, thereby allowing easier attachment of the enteric casing. A person skilled in the art would be aware of suitable coatings and uses thereof Preferably the seal coat is made of an OPADRY coating, and particularly preferably it is Opadry White OY-S-28876.

In some embodiments, an example of an enteric-coated formulation as described in WO2005/021002, can comprise varying amounts of one or more of ketamine, or analogue or salt thereof and scopolamine (or an analogue or derivative or salt thereof). In that example, lactose monohydrate, microcrystalline cellulose, the active ingredient, the hydroxypropyl methyl cellulose and half of the croscarmellose sodium were screened into a 10 Liter Fielder high-shear blender (any suitable high shear blender could be used) and blended for 5 minutes at 300 rpm with the chopper off. The mixture was then granulated by the addition of about 750 ml water whilst continuing to blend. The granules were dried in a Glatt ⅗ fluid bed drier, screened by Comil into a Pharmatec 5 Liter bin blender and then blended with any lactose anhydrous given in the formula plus the remainder of the croscarmellose sodium over 5 minutes at 20 rpm. Magnesium stearate was screened into the blender and the mixing process continued for a further 1 minute at 10 rpm. The lubricated mix was compressed using a Riva Piccolla rotary tablet press fitted with 9.5 mm round normal convex punches (any suitable tablet press could be used). The sealcoat, and subsequently the enteric coat, are applied by spraying of an aqueous suspension of the coat ingredients in a Manesty 10 coater using parameters for the coating process as recommended by the manufacturers of the coating polymers (again, any suitable coater could be used).

Other enteric-coated preparations of this sort can be prepared by one skilled in the art, using these materials or their equivalents.

### Other Formulations and Routes of Administration

In some embodiments, the compositions described herein comprising a combination of ketamine, or analogue or salt thereof and scopolamine (or an analogue or derivative or salt thereof) for use as a medicament, methods for preparing the medicament and methods for the sustained release of the medicament in vivo. Delivery systems can include time-release, delayed release or sustained release delivery systems, or as a pro-drug composition. Such systems can avoid repeated administrations of a pharmaceutical composition comprising a combination of ketamine, or analogue or salt thereof and an scopolamine (or an analogue or derivative or salt thereof) to increase convenience to the subject and/or the physician.

Many types of release delivery systems are available and known to those of ordinary skill in the art. They include, but are not limited to, polymer-based systems such as polylactic and polyglycolic acid, poly(lactide-glycolide), copolyoxalates, polyanhydrides, polyesteramides, polyorthoesters, polyhydroxybutyric acid, and polycaprolactone. Microcapsules of the foregoing polymers containing drugs are described in, for example, U.S. Pat. No. 5,075,109. Nonpolymer systems that are lipids including sterols such as cholesterol, cholesterol esters and fatty acids or neutral fats such as mono-, di- and tri-glycerides; phospholipids; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

In one embodiment, a polymeric matrix can be used for sustained delivery of the composition(s) described herein. Some examples for use of the polymeric matrix for containing the compounds of the invention include films, coatings, gels, implants, and stents. The size and composition of the polymeric matrix device is selected to result in favorable release kinetics in the tissue into which the matrix device is implanted. The size of the polymeric matrix device further is selected according to the method of delivery that is to be used. The polymeric matrix composition can be selected to have both favorable degradation rates and also to be formed of a material that is bioadhesive, to further increase the effectiveness of transfer when the device is administered to a vascular surface. The matrix composition also can be selected not to degrade, but rather, to release by diffusion over an extended period of time.

Both non-biodegradable and biodegradable polymeric matrices can be used to deliver agents and compounds as described herein to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multi-valent ions or other polymers.

Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof In general, these materials degrade either by enzymatic hydrolysis or exposure to water in vivo, by surface or bulk erosion.

Bioadhesive polymers of particular interest include bioerodible hydrogels may include, but are not limited to: polyhyaluronic acids, casein, gelatin, gluten, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Use of a long-term sustained release implant comprising a composition comprising a combination of ketamine, or analogue or salt thereof and scopolamine (or an analogue or derivative or salt thereof) inhibitor as disclosed herein can be particularly suitable for treatment of subjects with a depressive disorder, as well as subjects at risk of developing a such a disease or disorder.

The “long-term” release, as used herein, means that the implant is constructed and arranged to deliver therapeutic levels of composition comprising a combination of ketamine, or analogue or salt thereof and scopolamine (or an analogue or derivative or salt thereof) as disclosed herein for at least about 7 days, and in some embodiments about 30-60 days, and in some embodiments, for 4-6 months, or for 6-12 months, or longer than 12 months, for example, several years. Long-term sustained release implants are well known to those of ordinary skill in the art and include some of the release systems described above.

In some embodiments, the compositions as disclosed herein is administered to a subject using an infusion pump (to infuse, for example, the compositions as disclosed herein into the subject's circulatory system) is generally used intravenously, although subcutaneous, arterial, and epidural infusions are occasionally used. Injectable forms of administration are sometimes preferred for maximal effect. When long-term administration by injection is necessary, medi-ports, in-dwelling catheters, or automatic pumping mechanisms are also preferred, wherein direct and immediate access is provided to the arteries in and around the heart and other major organs and organ systems.

In some embodiments, compositions as disclosed herein comprising a combination of ketamine, or an analogue, derivative thereof and scopolamine (or an analogue or derivative or salt thereof) inhibitor can be administered to a specific site may be by transdermal transfusion, such as with a transdermal patch, by direct contact to the cells or tissue, if accessible, or by administration to an internal site through an incision or some other artificial opening into the body.

Production of a composition comprising a combination of ketamine, or an analogue, derivative thereof with scopolamine (or an analogue or derivative or salt thereof) inhibitor as disclosed herein according to present regulations will be regulated for good laboratory practices (GLP) and good manufacturing practices (GMP) by governmental agencies (e.g., U.S. Food and Drug Administration). This requires accurate and complete record keeping, as well as monitoring of QA/QC. Oversight of patient protocols by agencies and institutional panels is also envisioned to ensure that informed consent is obtained; safety, bioactivity, appropriate dosage, and efficacy of products are studied in phases; results are statistically significant; and ethical guidelines are followed. Similar oversight of protocols using animal models, as well as the use of toxic chemicals, and compliance with regulations is required.

In some embodiments, compositions of a combination of ketamine, or an analogue, derivative thereof with scopolamine (or an analogue or derivative or salt thereof) inhibitor as disclosed herein are also safe at effective dosages. Safe compositions are compositions that are not substantially toxic (e.g. cytotoxic or myelotoxic), or mutagenic at required dosages, do not cause adverse reactions or side effects, and are well-tolerated. Although side effects may occur, compositions are substantially safe if the benefits achieved from their use outweigh disadvantages that may be attributable to side effects. Unwanted side effects may include, but may not occur, frequent and/or sustained erections, nausea, vomiting, aggression, muscle development, baldness, hypersensitivity, allergic reactions, cardiovascular problems and other problems.

In some embodiments, the combination of ketamine, or an analogue, derivative thereof with scopolamine (or an analogue or derivative or salt thereof) inhibitor as disclosed herein can be administered to an adult, an adolescent, a child, in some embodiments, although rarely, the subject can be a neonate, an infant or in utero.

In some embodiments, the combination of ketamine, or an analogue, derivative thereof with scopolamine (or an analogue or derivative or salt thereof) inhibitor as disclosed herein can be administered according to a specific dosing regimen, e.g., in a single or multiple doses, or continuous or sporadic, or as deemed necessary based on an administration regime as determined by measuring total ketamine levels and/or free ketamine levels in the subject as disclosed herein.

In some embodiments, a combination of ketamine, or an analogue, derivative thereof with scopolamine, or an analogue, as disclosed herein can be administered to a subject via a continuous infusion throughout the cycle of therapy. Alternatively, a combination of ketamine, or an analogue, derivative thereof with scopolamine, or an analogue, as disclosed herein can be administered to a the subject over a single span of a few to several hours per day every day throughout the first period of the cycle of therapy.

Alternatively, in some embodiments a combination of ketamine, or an analogue, derivative thereof with scopolamine, or an analogue, as disclosed herein can be administered to a subject in a single parenteral bolus, or orally, daily for several days throughout the treatment regimen or cycle, or weekly.

Compositions as disclosed herein comprising a combination of ketamine, or an analogue, derivative thereof with scopolamine, or an analogue thereof, as disclosed herein can be physiologically stable at therapeutically effective concentrations. Physiological stable compounds of a combination of ketamine, or an analogue, derivative thereof with scopolamine, or an analogue thereof, as disclosed herein not break down or otherwise become ineffective upon administration to a subject or prior to having a desired effect. A combination of ketamine, or an analogue, derivative thereof with scopolamine, or an analogue thereof, inhibitor as disclosed herein can be structurally resistant to catabolism, and, thus, physiologically stable, or coupled by electrostatic or covalent bonds to specific reagents to increase physiological stability. Such reagents include amino acids such as arginine, glycine, alanine, asparagine, glutamine, histidine, or lysine, nucleic acids including nucleosides or nucleotides, or substituents such as carbohydrates, saccharides and polysaccharides, lipids, fatty acids, proteins, or protein fragments. Useful coupling partners include, for example, glycol, such as polyethylene glycol, glucose, glycerol, glycerin, and other related substances.

In some embodiments, a combination of ketamine, or an analogue, derivative thereof with scopolamine, or an analogue, as disclosed herein can additionally comprise chemicals that are substantially non-toxic. Substantially non-toxic means that the composition, although possibly possessing some degree of toxicity, is not harmful to the long-term health of the subject. Although the active component of the composition may not be toxic at the required levels, there may also be problems associated with administering the necessary volume or amount of the final form of the composition to the patient. For example, if a combination of ketamine, or an analogue, derivative thereof and scopolamine, or an analogue thereof, contains a salt, although the active ingredient may be at a concentration that is safe and effective, there can be a harmful build-up of sodium, potassium, or another ion. With a reduced requirement for the composition or at least the active component of that composition, the likelihood of such problems can be reduced or even eliminated. Consequently, although subjects may suffer minor or short term detrimental side-effects, the advantages of taking the composition outweigh the negative consequences.

In some embodiments, administration of a combination of ketamine, or an analogue, derivative thereof with scopolamine, or an analogue thereof, as disclosed herein can be intermittent; for example, administration can be once every two days, every three days, every five days, once a week, once or twice a month, and the like. The amount, forms, and/or amounts of the different forms of a combination of ketamine, or an analogue, derivative thereof with scopolamine, or an analogue thereof, can be varied at different times of administration.

### Administration and Efficacy

Dosages, formulations, dosage volumes, regimens, and methods for analyzing results of increasing the therapeutic levels of ketamine in a subject can vary. Thus, minimum and maximum effective dosages of a combination of ketamine, or an analogue, derivative thereof with scopolamine (or an analogue or derivative or salt thereof) inhibitor as disclosed herein vary depending on the method of administration. Ketamine or scopolamine levels in a subject can occur within a specific dosage range, which varies depending on, for example, the race, sex, gender, age, and overall health of the subject receiving the dosage, the route of administration, whether a composition comprising a combination of ketamine, or an analogue, derivative thereof with scopolamine (or an analogue or derivative or salt thereof) inhibitor as disclosed herein is administered in conjunction with other molecules, and the specific regimen of administration. For example, in general, nasal administration requires a smaller dosage than oral, enteral, rectal, or vaginal (if being administered to female) administration.

Suitable choices in amounts and timing of doses, formulation, and routes of administration of a combination of ketamine, or an analogue, derivative thereof with scopolamine (or an analogue or derivative or salt thereof) inhibitor as disclosed herein can be made with the goals of achieving a reduction in at least one symptom of depression or a related disorder.

A bolus of the formulation of a combination of ketamine, or an analogue, derivative thereof with scopolamine (or an analogue or derivative or salt thereof) inhibitor as disclosed herein can be administered to a subject over a short time period, for example, once a day is a convenient dosing schedule. Alternatively, an effective daily dose can be divided into multiple doses for purposes of administration, for example, two to twelve doses per day. Dosage levels of active ingredients in a pharmaceutical composition comprising a combination of ketamine, or an analogue, derivative thereof with scopolamine (or an analogue or derivative or salt thereof) inhibitor as disclosed herein can also be varied so as to achieve a transient or sustained concentration of the compound or derivative thereof in an individual, especially in and around the blood circulation and to result in the desired therapeutic response or protection. But it is also within the skill of the art to start doses at levels lower than required to achieve the desired therapeutic effect and to gradually increase the dosage until the desired effect is achieved.

In some embodiments, the amount of ketamine, or an analogue, derivative thereof with a scopolamine (or an analogue or derivative or salt thereof) inhibitor as disclosed herein to be administered is dependent upon factors known to a person skilled in the art such as bioactivity and bioavailability of the compound (e.g., half-life in the body, stability, and metabolism); chemical properties of the compound (e.g., molecular weight, hydrophobicity, and solubility); route and scheduling of administration, and the like. It will also be understood that the specific dose level to be achieved for any particular individual can depend on a variety of factors, including age, gender, health, medical history, weight, combination with one or more other drugs, and severity of disease.

The amount of a combination of ketamine, or an analogue, derivative thereof with scopolamine, or an analogue thereof, as disclosed herein that can be combined with a carrier material to produce a single dosage form will generally be that amount of the compound that produces a therapeutic effect. Generally out of one hundred percent, this amount will range from about 0.01% to 99% of the compound, preferably from about 5% to about 70%, most preferably from 10% to about 30%.

The data obtained from the cell culture assays and animal studies can be used in formulating a range of dosage for use in humans. The dosage of such compounds lies preferably within a range of circulating concentrations that include the ED₅₀ (the dose therapeutically effective in 50% of the population) with little or no toxicity. The dosage may vary within this range depending upon the dosage form employed and the route of administration utilized.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the combination of ketamine, or an analogue, derivative thereof with scopolamine, or an analogue thereof A desired dose can be administered every day or every third, fourth, fifth, or sixth day. The desired dose can be administered at one time or divided into sub-doses, e.g., 2-4 sub-doses and administered over a period of time, e.g., at appropriate intervals through the day or other appropriate schedule. Such sub-doses can be administered as unit dosage forms. In some embodiments of the aspects described herein, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

The terms “treatment” and “treating” as used herein, with respect to treatment of a disease, means preventing the progression of the disease, or altering the course of the disorder (for example, but are not limited to, slowing the progression of the disorder), or partially reversing a symptom of the disorder or reducing one or more symptoms and/or one or more biochemical markers in a subject, preventing one or more symptoms from worsening or progressing, promoting recovery or improving prognosis. For example, in the case of treating depression, therapeutic treatment refers to clinically relevant alleviation of at least one symptom associated with depression. Measurable lessening includes any clinically significant decline in a measurable marker or symptom, such as measuring markers for depression in the blood as described later or assessing the degree of depression, e.g., using the criteria listed in DSM-IV or the efficacy measures as described in the Examples, e.g., Hamilton Depression Rating Scale (HAMD, Hedlund J L, Viewig B W (1979) The Hamilton rating scale for depression: a comprehensive review. Journal of Operational Psychiatry 10:149-165) such as HAMD-17, HAMD-28, or HAMD-7 or CGI or Maier, after treatment. One can also use other scales, such as the Montgomery-Åsberg Depression Rating Scale (MADRS), the Beck Depression Inventory (BDI), the Zung Self-Rating Depression Scale, the Wechsler Depression Rating Scale, the Raskin Depression Rating Scale, the Inventory of Depressive Symptomatology (IDS), and the Quick Inventory of Depressive Symptomatology (QIDS). Clinicians are well aware of the various scales for measuring depression and can use any one of them to determine the severity of depression and whether the depression appears to be treatment resistant. For example, a score of 0-7 on HAMD is typically considered to be normal. Scores of 20 or higher indicate moderate, severe, or very severe depression. Questions 18-21 may be recorded to give further information about the depression (such as whether diurnal variation or paranoid symptoms are present), but are not necessary part of the scale. Thus, a reduction of symptoms can be considered clinically relevant if, e.g., the HAMD score is decreased under, e.g., 20. In one embodiment, at least one symptom of depression is alleviated by a “clinically relevant amount” as evaluated by a physician or a psychologist, as compared to a control (e.g., a subject having the same or similar degree of depression as the treated subject is administered without ketamine or scopolamine, or a subject who has met none of the conditions described herein is administered with treatment regimen comprising a ketamine and scopolamine). For example, in some embodiments, at least one neuropsychological test is improved (e.g., HAMD-17 rating is decreased) by at least about 10%, at least about 15%, at least about 20%, at least about 30%, at least about 40%, or at least about 50%. In another embodiment, at least one neuropsychological test is improved (e.g., HAMD-17 rating is decreased) by more than 50%, e.g., at least about 60%, or at least about 70%. In one embodiment, at least one neuropsychological test is improved (e.g., HAMD-17 rating is decreased) by at least about 80%, at least about 90% or greater, as compared to a control (e.g., a subject having the same or similar degree of depression as the treated subject is administered without treatment as described herein, or a subject who has met none of the conditions described herein is administered with treatment regimen comprising ketamine or scopolamine) In some embodiments, at least one symptom of depression can be alleviated by a clinically relevant amount as evaluated by a physician or a psychologist within a treatment period of at least about 10 days, including, e.g., at least about 20 days, at least about 30 days, at least about 40 days, or longer. In some embodiments, at least one neuropsychological test is improved (e.g., HAMD-17 rating is decreased) by at least about 10%, at least about 15%, at least about 20%, at least about 30%, at least about 40%, or at least about 50% or higher within a treatment period of at least about 10 days, including, e.g., at least about 20 days, at least about 30 days, at least about 40 days, or longer. In some embodiments of this aspect and all other aspects described herein, ketamine and scopolamine can be administered in an amount effective to reduce at least one symptom (e.g., but not limited to, low mood, anhedonia, low energy, insomnia, agitation, anxiety and/or weight loss) associated with depression, e.g., major depressive disorders.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference and may be employed in the practice of the invention. More generally, documents or references are cited in this text, either in a Reference List before the claims, or in the text itself; and, each of these documents or references (“herein cited references”), as well as each document or reference cited in each of the herein cited references (including any manufacturer's specifications, instructions, etc.), is hereby expressly incorporated herein by reference.

The invention can be understood more fully by reference to the following detailed description and illustrative examples, that are intended to exemplify non-limiting embodiments of the invention.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### EXAMPLES

Background: Scopolamine, a muscarinic cholinergic receptor antagonist, and ketamine, an NMDA receptor antagonist, have different effects on neuronal processes including memory, conditioned nictitating membrane response and cerebral vasodilation. Scopolamine produces a dose×difficulty related impairment of both recognition memory and incremental acquisition aspects of task performance, whereas ketamine administration results in a dose-dependent impairment of recognition memory but not incremental acquisition (Taffe MA, et al. Psychopharmacology (Berl). 2002 Mar; 160(3): 253-62). Scavio et al. examined whether post-training deliveries of scopolamine and ketamine modified the performance of the rabbit's conditioned nictitating membrane response (NMR) during acquisition and extinction (Scavio MJ, et al. Behav Neurosci. 1992 Dec; 106(6): 900-8). The results show that ketamine accelerates, but scopolamine retards, conditioning when the drugs are injected immediately after the completion of daily training sessions. Finally, ketamine injection (1 mg/kg) induces a significant cerebral vasodilatation that is blocked by scopolamine (Reicher D, et al. Stroke. 1987 Mar-Apr; 18(2): 445-9). These studies suggest that cholinergic receptor antagonists such as scopolamine and NMDA receptor antagonists such as ketamine have clearly distinctive and different neurobiological effects.

In other instances, ketamine and scopolamine may share some neurobiological effects (Figallo E M, et al. Psychopharmacology (Berl). 1979 Mar 14; 61(1): 59-62; Contreras C M, et al. Bol Estud Med Biol. 1990 Jan-Jun; 38(1-2): 10-5; Zhai H, et al. Behav Pharmacol. 2008 May; 19(3): 211-6; and Mudo' et al. Epilepsia. 1996 Feb; 37(2): 198-207). Recent clinical studies have demonstrated that both ketamine and scopolamine produce a rapid antidepressant response (within hours) and appear to be effective in treatment-resistant depressed patients (Machado-Vieira R, et al. Pharmacol Ther. 2009 Aug; 123(2): 143-50; Mathew S J, et al. CNS Drugs. 2012 Mar 1; 26(3): 189-204; Furey M L and Drevets W C. Arch Gen Psychiatry. 2006 Oct; 63(10): 1121-9; Drevets W C and Furey M L. Biol Psychiatry. 2010 Mar 1; 67(5): 432-8). However, while ketamine's antidepressant action is thought to be related to its rapid activation of the mammalian target of rapamycin (mTOR) and to its increase in synaptogenesis in the prefrontal cortex (Duman R S, et al. Neuropharmacology. 2012 Jan; 62(1): 35-41. Epub 2011 Sep 2), scopolamine appears to suppress adult neurogenesis in rats (Kotani S, et al. Neuroscience. 2006 Oct 13; 142(2): 505-14), again supporting the view that cholinergic receptor antagonists such as scopolamine and NMDA receptor antagonists such as ketamine have clearly distinctive and different neurobiological mechanisms.

Both scopolamine and ketamine have significant effects in one of the most widely used animal models of depression, the forced swim test (Ji C X and Zhang J J. Yao Xue Xue Bao. 2011 Apr; 46(4): 400-5; Mancinelli A, et al. Eur J Pharmacol. 1988 Dec 13; 158(3): 199-205; Yilmaz et al. Pharmacol Biochem Behav. 2002 Jan-Feb; 71(1-2): 341-4; and Garcia et al. Prog Neuropsychopharmacol Biol Psychiatry. 2008 Jan 1; 32(1): 140-4). It has now been determined that scopolamine and ketamine have significant effects in the forced swim test when administered in dosages corresponding to subclinical doses individually.

Example 1

### Combination of Ketamine & Scopolamine Show Antidepressant Efficacy in the Mouse Forced Swim Test

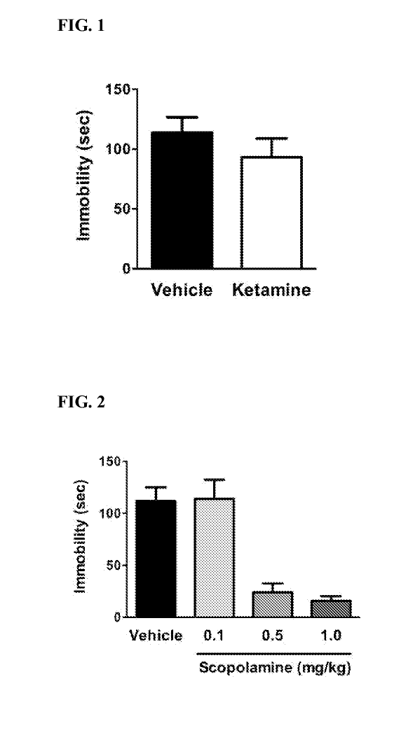

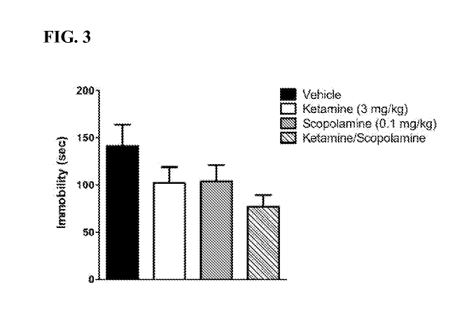

Summary Male 10 week C57BL/6N mice (Taconic) acclimated to a holding facility one week prior to testing. A two-day FST paradigm was employed, in which mice were exposed to a 10 minute test in absence of drug, and tested the following day in presence of drug in a 6 minute test. Scopolamine and ketamine were administered intraperitoneally (i.p.) in saline vehicle 30 minutes prior to FST. All animals were drug naive and not previously exposed to behavioral testing. As depicted in FIG. 1, ketamine at 3 mg/kg i.p. produces a modest non-significant antidepressant effect in the FST (N=6 mice/group). In FIG. 2, it is shown that scopolamine at 0.1 mg/kg does not produce an antidepressant effect in the mouse FST, with no change in immobility time versus vehicle. Scopolamine at 0.5 and 1.0 mg/kg substantially reduced immobility time, appearing to produce an antidepressant effect. However, the reduced immobility was likely due to motor activation since mice were noticeably hyperactive in the home cage after scopolamine administration prior to testing (N=6 mice/group). In FIG. 3, it is shown that scopolamine, administered at an ineffective dose (0.1 mg/kg i.p.), improves the modest non-significant antidepressant effect of ketamine (3 mg/kg i.p.), resulting in significantly reduced immobility versus vehicle (t-test p=0.016, N=10-11 mice/group).

### Materials and Methods

Animals: Male C57BL6N/Tac mice were purchased from Taconic Farms (Germantown, N.Y.) at 10 weeks of age. Mice were housed four mice per cage upon arrival under a 12:12 h light/dark cycle and were allowed to acclimate for approximately one week prior to behavioral procedures. Food and water were provided ad libitum. All procedures followed the National Institutes of Health Guide for the Care and Use of Laboratory Animals, and were approved by the Massachusetts Institute of Technology Animal Care and Use Committee.

Drug treatment: Ketamine hydrochloride and scopolamine hydrochloride were purchased from Sigma-Aldrich (St. Louis, Mo.) and were dissolved in saline vehicle (0.9% sodium chloride). For the scopolamine dose response experiment, mice received vehicle or 0.1, 0.5, or 1 mg/kg i.p. scopolamine 30 minutes prior to behavior testing. For the ketamine/scopolamine interaction experiment, mice received two i.p. injections 30 minutes prior to behavior testing or sacrifice for biochemical studies: vehicle/vehicle, vehicle/scopolamine (0.1 mg/kg), vehicle/ketamine (3 mg/kg), or scopolamine (0.1 mg/kg)/ketamine (3 mg/kg).

Behavioral procedures: Mice were tested in a two day forced swim test (FST) procedure. On day 1, all mice received mock intraperitoneal vehicle (i.p.) injections to acclimate to the injection procedure and, 30 minutes later, were placed into one of five identical cylindrical chambers (24 cm×15 cm) filled approximately halfway with warm water (26±2°) for 10 minutes with no data collected. On day 2, mice received i.p. injections 30 minutes prior to being placed in a cylinder for a 6 minute session and immobility time was scored automatically (Etho VisionXT; Noldus Information Technology; Wageningen, Netherlands) during the final 4 minutes.

Statistical analysis: For all FST behavioral experiments, the time spent immobile was the dependent variable, and data were analyzed by one-way analysis of variance (ANOVA) and Tukey post-hoc comparisons using SPSS v. 18 (IBM).

### Results

### Subeffective Doses of Ketamine and Scopolamine in the Mouse FST

The inventors initially identified doses of ketamine and scopolamine that alone are ineffective in producing significant antidepressant effects in the FST in mice. Prior studies of ketamine in mice and rats indicated that a dose between 3-10 mg/kg i.p. can produce antidepressant effects in the FST (Autry et al., 2011; Li et al., 2010). A dose at the low end of this range was selected (3 mg/kg i.p.) and administered to adult male C57BL/6N mice 30 min prior to the FST. Only a modest and non-significant 18% reduction in immobility was observed (mean +/−SE; vehicle 113.9 sec +/−12.7, ketamine 93.1 sec +/−15.9; FIG. 1). As there are few published studies of scopolamine in rodent depression models, a dose response was performed to identify a subeffective dose of scopolamine in C57BL/6N mice (FIG. 2). Scopolamine (0.1 mg/kg i.p.) administered 30 min prior to the FST did not significantly reduce immobility time versus vehicle (vehicle 112.1 sec +/−11.9, scopolamine 114.0sec +/−17.0; p>0.05). Higher scopolamine doses (0.5 mg/kg and 1.0 mg/kg i.p.) substantially reduced immobility time (p<0.05; FIG. 2); however, this was likely due to motor activation since mice were visibly hyperactive in the home cage immediately after scopolamine administration prior to the FST, in line with a previous report (Ji and Zhang, 2011).

### Ketamine and Scopolamine Interaction in the Mouse FST

Doses of ketamine (3 mg/kg i.p.) and scopolamine (0.1 mg/kg i.p.) were selected that alone were subeffective in the FST and the inventors evaluated whether co-treatment produced significant antidepressant effects in the FST. As in the experiments above, administration of ketamine (3 mg/kg i.p.; 102.4 sec +/−16.0) or scopolamine (0.1 mg/kg i.p.; 103.9 sec +/−16.3) did not significantly reduce FST immobility time compared to vehicle (141.6 sec +/−21.7) (both Tukey's post-hoc p>0.05; FIG. 3). In contrast, co-treatment with both drugs at these same doses significantly reduced immobility time (77.1 sec +/−12.0) compared to vehicle (Tukey's post-hoc p=0.016; FIG. 3).

From the foregoing description, it will be apparent that variations and modifications may be made to the invention described herein to adopt it to various usages and conditions. Such embodiments are also within the scope of the following claims. The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

### REFERENCES

All patents, patent applications and publications mentioned in this specification are herein incorporated by reference to the same extent as if each independent patent and publication was specifically and individually indicated to be incorporated by reference.
1. Kessler R C, Berglund P, Demler O, Jin R, Koretz D, Merikangas K R, Rush A J, Walters E E, Wang P S: The epidemiology of major depressive disorder: results from the National Comorbidity Survey Replication (NCS-R). Jama 2003; 289(23):3095-105
2. Rush A J, Warden D, Wisniewski S R, Fava M, Trivedi M H, Gaynes B N, Nierenberg A A: STAR*D: revising conventional wisdom. CNS Drugs 2009; 23(8): 627-47
3. Crown W H, Finkelstein S, Berndt E R, Ling D, Poret A W, Rush A J, Russell J M: The impact of treatment-resistant depression on health care utilization and costs. J Clin Psychiatry 2002; 63(11): 963-71
4. Hoyer E H, Mortensen P B, Olesen A V: Mortality and causes of death in a total national sample of patients with affective disorders admitted for the first time between 1973 and 1993. Br J Psychiatry 2000; 176: 76-82
5. Osby U, Brandt L, Correia N, Ekbom A, Sparen P: Excess mortality in bipolar and unipolar disorder in Sweden. Arch Gen Psychiatry 2001; 58(9): 844-50
6. Martin B A: The Clarke Institute experience with completed suicide: 1966 to 1997. Can J Psychiatry 2000; 45(7): 630-8
7. Holma K M, Melartin T K, Haukka J, Holma I A, Sokero T P, Isometsa E T: Incidence and predictors of suicide attempts in DSM-IV major depressive disorder: a five-year prospective study. Am J Psychiatry 2010; 167(7): 801-8
8. Oquendo M A, Kamali M, Ellis S P, Grunebaum M F, Malone K M, Brodsky B S, Sackeim H A, Mann J J: Adequacy of antidepressant treatment after discharge and the occurrence of suicidal acts in major depression: a prospective study. Am J Psychiatry 2002; 159(10): 1746-51
9. Chynoweth R, Tonge J I, Armstrong J: Suicide in Brisbane—a retrospective psychosocial study. Aust N Z J Psychiatry 1980; 14(1): 37-45
10. Asgard U: A psychiatric study of suicide among urban Swedish women. Acta Psychiatr Scand 1990; 82(2): 115-24
11. Andersen U A, Andersen M, Rosholm J U, Gram L F: Psychopharmacological treatment and psychiatric morbidity in 390 cases of suicide with special focus on affective disorders. Acta Psychiatr Scand 2001; 104(6): 458-65
12. Isometsa E, Henriksson M, Aro H, Heikkinen M, Kuoppasalmi K, Lonnqvist J: Suicide in psychotic major depression. J Affect Disord 1994; 31(3): 187-91
13. Isometsa E T, Aro H M, Henriksson M M, Heikkinen M E, Lonnqvist J K: Suicide in major depression in different treatment settings. J Clin Psychiatry 1994; 55(12): 523-7
14. Waern M, Beskow J, Runeson B, Skoog I: High rate of antidepressant treatment in elderly people who commit suicide. Bmj 1996; 313(7065): 1118
15. Kudoh A, Takahira Y, Katagai H, Takazawa T: Small-dose ketamine improves the postoperative state of depressed patients. Anesth Analg 2002; 95(1): 114-8, table of contents
16. Correll G E, Futter G E: Two case studies of patients with major depressive disorder given low-dose (subanesthetic) ketamine infusions. Pain Med 2006; 7(1): 92-5
17. Berman R M, Cappiello A, Anand A, Oren D A, Heninger G R, Charney D S, Krystal J H: Antidepressant effects of ketamine in depressed patients. Biol Psychiatry 2000; 47(4): 351-4
18. Zarate C A, Jr., Singh J B, Carlson P J, Brutsche N E, Ameli R, Luckenbaugh D A, Charney D S, Manji H K: A randomized trial of an N-methyl-D-aspartate antagonist in treatment-resistant major depression. Arch Gen Psychiatry 2006; 63(8): 856-64
19. Phelps L E, Brutsche N, Moral J R, Luckenbaugh D A, Manji H K, Zarate C A, Jr.: Family history of alcohol dependence and initial antidepressant response to an N-methyl-D-aspartate antagonist. Biol Psychiatry 2009; 65(2): 181-4
20. Price R B, Nock M K, Charney D S, Mathew S J: Effects of intravenous ketamine on explicit and implicit measures of suicidality in treatment-resistant depression. Biol Psychiatry 2009; 66(5): 522-6
21. van het Rot M, Collins K A, Murrough J W, Perez A M, Reich D L, Charney D S, Mathew S J: Safety and efficacy of repeated-dose intravenous ketamine for treatment-resistant depression. Biol Psychiatry 2010; 67(2): 139-45
22. Furey M L, Drevets W C: Antidepressant efficacy of the antimuscarinic drug scopolamine: a randomized, placebo-controlled clinical trial. Arch Gen Psychiatry 2006; 63(10): 1121-9
23. Olney J W, Labruyere J, Wang G, Wozniak D F, Price M T, Sesma M A: NMDA antagonist neurotoxicity: mechanism and prevention. Science 1991; 254(5037): 1515-8
24. Morita T, Hitomi S, Saito S, Fujita T, Uchihashi Y, Kuribara H: Repeated ketamine administration produces up-regulation of muscarinic acetylcholine receptors in the forebrain, and reduces behavioral sensitivity to scopolamine in mice. Psychopharmacology (Berl) 1995; 117(4): 396-402
25. Ji C X, Zhang J J: [Effect of scopolamine on depression in mice]. Yao Xue Xue Bao 2011; 46(4): 400-5
26. Mancinelli A, Borsini F, d'Aranno V, Lecci A, Meli A: Cholinergic drug effects on antidepressant—induced behavior in the forced swimming test. Eur J Pharmacol 1988; 158(3): 199-205
27. Yilmaz A, Schulz D, Aksoy A, Canbeyli R: Prolonged effect of an anesthetic dose of ketamine on behavioral despair. Pharmacol Biochem Behav 2002; 71(1-2): 341-4
28. Garcia L S, Comim C M, Valvassori S S, Reus G Z, Barbosa L M, Andreazza A C, Stertz L, Fries G R, Gavioli E C, Kapczinski F, Quevedo J: Acute administration of ketamine induces antidepressant-like effects in the forced swimming test and increases BDNF levels in the rat hippocampus. Prog Neuropsychopharmacol Biol Psychiatry 2008; 32(1): 140-4
29. Diazgranados N, Ibrahim L, Brutsche N E, Newberg A, Kronstein P, Khalife S, Kammerer W A, Quezado Z, Luckenbaugh D A, Salvadore G, Machado-Vieira R, Manji H K, Zarate C A, Jr.: A randomized add-on trial of an N-methyl-D-aspartate antagonist in treatment-resistant bipolar depression. Arch Gen Psychiatry 2010; 67(8): 793-802

1. A method of treating or reducing depression in a human subject the method comprising administering to the human subject an effective amount of about 500 mcg per kilogram of body weight per day or less of ketamine in combination with about 4 mcg per kilogram of body weight per day or less of scopolamine, thereby treating or reducing depression in the human subject.

2-21. (canceled)
